(19) United States
(12) Patent Application Publication
(10) Pub. No.: US 2021/0215651 A1
(43) Pub. Date: Jul. 15, 2021

(54) ESTIMATING UNKNOWN PROPORTIONS OF A PLURALITY OF END-MEMBERS IN AN UNKNOWN MIXTURE

(71) Applicant: CHEVRON U.S.A. INC., San Ramon, CA (US)

(72) Inventor: Lingbo XING, Houston, TX (US)

(73) Assignee: CHEVRON U.S.A. INC., San Ramon, CA (US)

(21) Appl. No.: 17/150,033

(22) Filed: Jan. 15, 2021

Related U.S. Application Data
(60) Provisional application No. 62/961,498, filed on Jan. 15, 2020.

(51) Int. Cl. G01N 30/86; G01N 33/24

(52) U.S. Cl. CPC G01N 30/8686; E21B 43/16; G01N 30/8637; G01N 33/241

(57) ABSTRACT

Embodiments of estimating unknown proportions of a plurality of end-members in an unknown mixture are provided herein. One embodiment of a method of estimating unknown proportions of a plurality of end-members in an unknown mixture comprises receiving fingerprint data of a plurality of end-members and an unknown mixture comprising unknown proportions of the plurality of end-members; processing the fingerprint data of the plurality of end-members and the unknown mixture to generate peak height data of the plurality of end-members and the unknown mixture; and generating an estimate of the unknown proportions of the plurality of end-members in the unknown mixture by applying a Markov Chain Monte Carlo method to the peak height data of the plurality of end-members and the unknown mixture.

### CROSS REFERENCES TO RELATED APPLICATIONS

This application claims the benefit of priority to 62/961,498 filed Jan. 15, 2020, which is incorporated by reference in its entirety.

### STATEMENT REGARDING FEDERALLY SPONSORED RESEARCH OR DEVELOPMENT

Not applicable.

### TECHNICAL FIELD

The present disclosure relates to estimating unknown proportions of end-members in an unknown mixture.

### BACKGROUND

The hydrocarbon industry recovers hydrocarbons, such as oils, that are trapped in subsurface reservoirs (also known as subsurface formations). The hydrocarbons can be recovered by drilling wellbores (also known as wells) into the reservoirs and the hydrocarbons are able to flow from the reservoirs into the wellbores and up to the surface. Commingling of downhole production from stacked reservoirs (also known as zones) is a common practice. Commingling has many benefits during the development of a field, including high production rates per well, reduced infrastructure, reduced capital and operational costs, and a smaller environmental footprint.

Although commingling is common practice, it is beneficial to perform zonal allocation for effective well and reservoir management. For example, oils from a single reservoir have a nearly identical fingerprint, whereas oils from separate reservoirs usually have consistent fingerprint differences. The contribution of each reservoir/flowline oil to the commingled oil flow can be calculated based on the identified fingerprint differences. Unfortunately, the allocation process was mostly solved using constrained least square methods with the assumption of linear mixing behavior, which requires lab mixed samples with a known mixture as calibration to optimize the fingerprint parameters selection process. Moreover, end-member samples are oftentimes not sufficient to make the required known mixture.

Thus, there exists a need in estimating unknown proportions of end-members in an unknown mixture.

### SUMMARY

Embodiments of estimating unknown proportions of a plurality of end-members and an unknown mixture are provided herein. One embodiment of a method of estimating unknown proportions of a plurality of end-members in an unknown mixture comprises receiving fingerprint data of a plurality of end-members and an unknown mixture comprising unknown proportions of the plurality of end-members; processing the fingerprint data of the plurality of end-members and the unknown mixture to generate peak height data of the plurality of end-members and the unknown mixture; and generating an estimate of the unknown proportions of the plurality of end-members in the unknown mixture by applying a Markov Chain Monte Carlo method to the peak height data of the plurality of end-members and the unknown mixture.

One embodiment of a system comprises a processor and a memory communicatively connected to the processor, the memory storing computer-executable instructions which, when executed, cause the processor to perform a method of estimating unknown proportions of a plurality of end-members in an unknown mixture. The method comprising receiving fingerprint data of a plurality of end-members and an unknown mixture comprising unknown proportions of the plurality of end-members; processing the fingerprint data of the plurality of end-members and the unknown mixture to generate peak height data of the plurality of end-members and the unknown mixture; and generating an estimate of the unknown proportions of the plurality of end-members in the unknown mixture by applying a Markov Chain Monte Carlo method to the peak height data of the plurality of end-members and the unknown mixture.

One embodiment of a computer readable storage medium having computer-executable instructions stored thereon which, when executed by a computer, cause the computer to perform a method of estimating unknown proportions of a plurality of end-members in an unknown mixture. The method comprising receiving fingerprint data of a plurality of end-members and an unknown mixture comprising unknown proportions of the plurality of end-members; processing the fingerprint data of the plurality of end-members and the unknown mixture to generate peak height data of the plurality of end-members and the unknown mixture; and generating an estimate of the unknown proportions of the plurality of end-members in the unknown mixture by applying a Markov Chain Monte Carlo method to the peak height data of the plurality of end-members and the unknown mixture.

### DESCRIPTION OF THE DRAWINGS

FIG. 1 illustrates one embodiment of a system of estimating unknown proportions of a plurality of end-members in an unknown mixture.

FIG. 2 illustrates one embodiment of a method of estimating unknown proportions of a plurality of end-members in an unknown mixture.

FIG. 3 illustrates one example of fingerprint data.

FIGS. 4A, 4B, and 4C illustrate examples of processing the fingerprint data of the plurality of end-members and the unknown mixture to generate peak height data of the plurality of end-members and the unknown mixture. Specifically, FIGS. 4A, 4B, and 4C (top) illustrate an example of peak alignment and FIGS. 4A, 4B, and 4C (bottom) illustrates an example of indexing.

FIG. 5 illustrates examples of generating an estimate of the unknown proportions of the plurality of end-members in the unknown mixture by applying a Markov Chain Monte Carlo method to the peak height data of the plurality of end-members and the unknown mixture.

FIG. 6A illustrates an example of the similarity of the oils from 2 zones.

FIG. 6B illustrates examples of a difference of 0% to 6% based on a comparison of the generated estimate to proportions generated by well test data.

FIG. 7 illustrates one example of validation using lab mixed samples with 4 end-members oil from the stacked reservoir of a single well.

Reference will now be made in detail to various embodiments, where like reference numerals designate corresponding parts throughout the several views. In the following detailed description, numerous specific details are set forth in order to provide a thorough understanding of the present disclosure and the embodiments described herein. However, embodiments described herein may be practiced without these specific details. In other instances, well-known methods, procedures, components, and mechanical apparatuses have not been described in detail so as not to unnecessarily obscure aspects of the embodiments.

### DETAILED DESCRIPTION

TERMINOLOGY: The following terms will be used throughout the specification and will have the following meanings unless otherwise indicated.

Formation: Hydrocarbon exploration processes, hydrocarbon recovery (also referred to as hydrocarbon production) processes, or any combination thereof may be performed on a formation. The formation refers to practically any volume under a surface. For example, the formation may be practically any volume under a terrestrial surface (e.g., a land surface), practically any volume under a seafloor, etc. A water column may be above the formation, such as in marine hydrocarbon exploration, in marine hydrocarbon recovery, etc. The formation may be onshore. The formation may be offshore (e.g., with shallow water or deep water above the formation). The formation may include faults, fractures, overburdens, underburdens, salts, salt welds, rocks, sands, sediments, pore space, etc. Indeed, the formation may include practically any geologic point(s) or volume(s) of interest (such as a survey area) in some embodiments.

The formation may include hydrocarbons, such as liquid hydrocarbons (also known as oil or petroleum), gas hydrocarbons (e.g., natural gas), solid hydrocarbons (e.g., asphaltenes or waxes), a combination of hydrocarbons (e.g., a combination of liquid hydrocarbons and gas hydrocarbons) (e.g., a combination of liquid hydrocarbons, gas hydrocarbons, and solid hydrocarbons), etc. Light crude oil, medium oil, heavy crude oil, and extra heavy oil, as defined by the American Petroleum Institute (API) gravity, are examples of hydrocarbons. Examples of hydrocarbons are many, and hydrocarbons may include oil, natural gas, kerogen, bitumen, etc. The hydrocarbons may be discovered by hydrocarbon exploration processes.

The formation may also include at least one wellbore. For example, at least one wellbore may be drilled into the formation in order to confirm the presence of the hydrocarbons. As another example, at least one wellbore may be drilled into the formation in order to recover (also referred to as produce) the hydrocarbons. The hydrocarbons may be recovered from the entire formation or from a portion of the formation. For example, the formation may be divided into one or more hydrocarbon zones, and hydrocarbons may be recovered from each desired hydrocarbon zone. One or more of the hydrocarbon zones may even be shut-in to increase hydrocarbon recovery from a hydrocarbon zone that is not shut-in.

The formation, the hydrocarbons, or any combination thereof may also include non-hydrocarbon items. For example, the non-hydrocarbon items may include connate water, brine, tracers, items used in enhanced oil recovery or other hydrocarbon recovery processes, etc.

In short, each formation may have a variety of characteristics, such as petrophysical rock properties, reservoir fluid properties, reservoir conditions, hydrocarbon properties, or any combination thereof. For example, each formation (or even zone or portion of the formation) may be associated with one or more of: temperature, porosity, salinity, permeability, water composition, mineralogy, hydrocarbon type, hydrocarbon quantity, reservoir location, pressure, etc. Indeed, those of ordinary skill in the art will appreciate that the characteristics are many, including, but not limited to: shale gas, shale oil, tight gas, tight oil, tight carbonate, carbonate, vuggy carbonate, unconventional (e.g., a rock matrix with an average pore size less than 1 micrometer), diatomite, geothermal, mineral, metal, a formation having a permeability in the range of from 0.000001 millidarcy to 25 millidarcy (such as an unconventional formation), a formation having a permeability in the range of from 26 millidarcy to 40,000 millidarcy, etc.

The terms “formation”, “subsurface formation”, “hydrocarbon-bearing formation”, “reservoir”, “subsurface reservoir”, “subsurface region of interest”, “subterranean reservoir”, “subsurface volume of interest”, “subterranean formation”, and the like may be used synonymously. The terms “formation”, “hydrocarbons”, and the like are not limited to any description or configuration described herein.

Wellbore: A wellbore refers to a single hole, usually cylindrical, that is drilled into the formation for hydrocarbon exploration, hydrocarbon recovery, surveillance, or any combination thereof. The wellbore is usually surrounded by the formation and the wellbore may be configured to be in fluidic communication with the formation (e.g., via perforations). The wellbore may also be configured to be in fluidic communication with the surface, such as in fluidic communication with a surface facility that may include oil/gas/water separators, gas compressors, storage tanks, pumps, gauges, sensors, meters, pipelines, etc.

The wellbore may be used for injection (sometimes referred to as an injection wellbore) in some embodiments. The wellbore may be used for production (sometimes referred to as a production wellbore) in some embodiments. The wellbore may be used for a single function, such as only injection, in some embodiments. The wellbore may be used for a plurality of functions, such as production then injection, in some embodiments. The use of the wellbore may also be changed, for example, a particular wellbore may be turned into an injection wellbore after a different previous use as a production wellbore. The wellbore may be drilled amongst existing wellbores, for example, as an infill wellbore. A wellbore may be utilized for injection and a different wellbore may be used for hydrocarbon production, such as in the scenario that hydrocarbons are swept from at least one injection wellbore towards at least one production wellbore and up the at least one production wellbore towards the surface for processing. On the other hand, a single wellbore may be utilized for injection and hydrocarbon production, such as a single wellbore used for hydraulic fracturing and hydrocarbon production. A plurality of wellbores (e.g., tens to hundreds of wellbores) are often used in a field to recover hydrocarbons.

The wellbore may have straight, directional, or a combination of trajectories. For example, the wellbore may be a vertical wellbore, a horizontal wellbore, a multilateral wellbore, an inclined wellbore, a slanted wellbore, etc. The wellbore may include a change in deviation. As an example, the deviation is changing when the wellbore is curving. In a horizontal wellbore, the deviation is changing at the curved section (sometimes referred to as the heel). As used herein, a horizontal section of a wellbore is drilled in a horizontal direction (or substantially horizontal direction). For example, a horizontal section of a wellbore is drilled towards the bedding plane direction. On the other hand, a vertical wellbore is drilled in a vertical direction (or substantially vertical direction). For example, a vertical wellbore is drilled perpendicular (or substantially perpendicular) to the bedding plane direction.

The wellbore may include a plurality of components, such as, but not limited to, a casing, a liner, a tubing string, a heating element, a sensor, a packer, a screen, a gravel pack, artificial lift equipment (e.g., an electric submersible pump (ESP)), etc. The “casing” refers to a steel pipe cemented in place during the wellbore construction process to stabilize the wellbore. The “liner” refers to any string of casing in which the top does not extend to the surface but instead is suspended from inside the previous casing. The “tubing string” or simply “tubing” is made up of a plurality of tubulars (e.g., tubing, tubing joints, pup joints, etc.) connected together. The tubing string is lowered into the casing or the liner for injecting a fluid into the formation, producing a fluid from the formation, or any combination thereof. The casing may be cemented in place, with the cement positioned in the annulus between the formation and the outside of the casing. The wellbore may also include any completion hardware that is not discussed separately. If the wellbore is drilled offshore, the wellbore may include some of the previous components plus other offshore components, such as a riser.

The wellbore may also include equipment to control fluid flow into the wellbore, control fluid flow out of the wellbore, or any combination thereof. For example, each wellbore may include a wellhead, a BOP, chokes, valves, or other control devices. These control devices may be located on the surface, under the surface (e.g., downhole in the wellbore), or any combination thereof. In some embodiments, the same control devices may be used to control fluid flow into and out of the wellbore. In some embodiments, different control devices may be used to control fluid flow into and out of the wellbore. In some embodiments, the rate of flow of fluids through the wellbore may depend on the fluid handling capacities of the surface facility that is in fluidic communication with the wellbore. The control devices may also be utilized to control the pressure profile of the wellbore.

The equipment to be used in controlling fluid flow into and out of the wellbore may be dependent on the wellbore, the formation, the surface facility, etc. However, for simplicity, the term “control apparatus” is meant to represent any wellhead(s), BOP(s), choke(s), valve(s), fluid(s), and other equipment and techniques related to controlling fluid flow into and out of the wellbore.

The wellbore may be drilled into the formation using practically any drilling technique and equipment known in the art, such as geosteering, directional drilling, etc. Drilling the wellbore may include using a tool, such as a drilling tool that includes a drill bit and a drill string. Drilling fluid, such as drilling mud, may be used while drilling in order to cool the drill tool and remove cuttings. Other tools may also be used while drilling or after drilling, such as measurement-while-drilling (MWD) tools, seismic-while-drilling (SWD) tools, wireline tools, logging-while-drilling (LWD) tools, or other downhole tools. After drilling to a predetermined depth, the drill string and the drill bit are removed, and then the casing, the tubing, etc. may be installed according to the design of the wellbore.

The equipment to be used in drilling the wellbore may be dependent on the design of the wellbore, the formation, the hydrocarbons, etc. However, for simplicity, the term “drilling apparatus” is meant to represent any drill bit(s), drill string(s), drilling fluid(s), and other equipment and techniques related to drilling the wellbore.

The term “wellbore” may be used synonymously with the terms “borehole,” “well,” or “well bore.” The term “wellbore” is not limited to any description or configuration described herein.

Hydrocarbon recovery: The hydrocarbons may be recovered (sometimes referred to as produced) from the formation using primary recovery (e.g., by relying on pressure to recover the hydrocarbons), secondary recovery (e.g., by using water injection (also referred to as waterflooding) or natural gas injection to recover hydrocarbons), enhanced oil recovery (EOR), or any combination thereof. Enhanced oil recovery or simply EOR refers to techniques for increasing the amount of hydrocarbons that may be extracted from the formation. Enhanced oil recovery may also be referred to as tertiary oil recovery. Secondary recovery is sometimes just referred to as improved oil recovery or enhanced oil recovery. EOR processes include, but are not limited to, for example: (a) miscible gas injection (which includes, for example, carbon dioxide flooding), (b) chemical injection (sometimes referred to as chemical enhanced oil recovery (CEOR) that includes, for example, polymer flooding, alkaline flooding, surfactant flooding, conformance control, as well as combinations thereof such as alkaline-polymer (AP) flooding, surfactant-polymer (SP) flooding, or alkaline-surfactant-polymer (ASP) flooding), (c) microbial injection, (d) thermal recovery (which includes, for example, cyclic steam and steam flooding), or any combination thereof. The hydrocarbons may be recovered from the formation using a fracturing process. For example, a fracturing process may include fracturing using electrodes, fracturing using fluid (oftentimes referred to as hydraulic fracturing), etc. The hydrocarbons may be recovered from the formation using radio frequency (RF) heating. Another hydrocarbon recovery process(s) may also be utilized to recover the hydrocarbons. Furthermore, those of ordinary skill in the art will appreciate that one hydrocarbon recovery process may also be used in combination with at least one other recovery process or subsequent to at least one other recovery process. This is not an exhaustive list of hydrocarbon recovery processes.

Other definitions: The term “proximate” is defined as “near”. If item A is proximate to item B, then item A is near item B. For example, in some embodiments, item A may be in contact with item B. For example, in some embodiments, there may be at least one barrier between item A and item B such that item A and item B are near each other, but not in contact with each other. The barrier may be a fluid barrier, a non-fluid barrier (e.g., a structural barrier), or any combination thereof. Both scenarios are contemplated within the meaning of the term “proximate.”

The terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a” or “an” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) includes 10% and also includes 20%, and includes percentages in between 10% and 20%, unless explicitly stated otherwise herein. Similarly, a range of between 10% and 20% (i.e., range between 10%-20%) includes 10% and also includes 20%, and includes percentages in between 10% and 20%, unless explicitly stated otherwise herein.

The term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein. By way of example, if an item is described herein as including a component of type A, a component of type B, a component of type C, or any combination thereof, it is understood that this phrase describes all of the various individual and collective combinations and permutations of these components. For example, in some embodiments, the item described by this phrase could include only a component of type A. In some embodiments, the item described by this phrase could include only a component of type B. In some embodiments, the item described by this phrase could include only a component of type C. In some embodiments, the item described by this phrase could include a component of type A and a component of type B. In some embodiments, the item described by this phrase could include a component of type A and a component of type C. In some embodiments, the item described by this phrase could include a component of type B and a component of type C. In some embodiments, the item described by this phrase could include a component of type A, a component of type B, and a component of type C. In some embodiments, the item described by this phrase could include two or more components of type A (e.g., A1 and A2). In some embodiments, the item described by this phrase could include two or more components of type B (e.g., B1 and B2). In some embodiments, the item described by this phrase could include two or more components of type C (e.g., C1 and C2). In some embodiments, the item described by this phrase could include two or more of a first component (e.g., two or more components of type A (A1 and A2)), optionally one or more of a second component (e.g., optionally one or more components of type B), and optionally one or more of a third component (e.g., optionally one or more components of type C). In some embodiments, the item described by this phrase could include two or more of a first component (e.g., two or more components of type B (B1 and B2)), optionally one or more of a second component (e.g., optionally one or more components of type A), and optionally one or more of a third component (e.g., optionally one or more components of type C). In some embodiments, the item described by this phrase could include two or more of a first component (e.g., two or more components of type C (C1 and C2)), optionally one or more of a second component (e.g., optionally one or more components of type A), and optionally one or more of a third component (e.g., optionally one or more components of type B).

This written description uses examples to disclose the invention, including the best mode, and also to enable any person skilled in the art to make and use the invention. The patentable scope is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

Unless defined otherwise, all technical and scientific terms used herein have the same meanings as commonly understood by one of skill in the art to which the disclosed invention belongs. All citations referred herein are expressly incorporated by reference.

An oil fingerprint is defined here as a series of hydrocarbon peak heights determined by whole oil Gas Chromatography (GC). The oil fingerprinting allocation approach is based on a well-established proposition that oils from the same reservoir exhibit nearly identical fingerprints, whereas oils from separate reservoir usually show measurable chromatographic differences. Allocation is the process of decomposing the oil fingerprints of a mixed/commingled oil into a set of end-member oils and their corresponding abundances. All the end-member fingerprints are assumed to be known. Traditionally, deterministic least square linear regression has been used on peak height ratios to fit the allocation model and provide a single best-fit abundance for multiple sources. Although this conventional approach has been successful, there are some drawbacks, especially its limit of 3 end members, its requirement for the calibration sets from lab mixed samples, and the difficulty with non-linearity of the equations derived from mixing and non-normal distributions of random errors to estimate confidence intervals. Moreover, the conventional approach has drawbacks in the context of deepwater environments, due to multiple stacked reservoirs, thick pay, and low permeability sands, and the development and production of reservoirs that will exist for decades. There are technical challenges with using the classic linear least square method on oil fingerprinting for long term production allocation in these types of developments: 1) subtle oil fingerprint differences between commingled zones, 2) more than 3 commingled zones, 3) compositional elucidation of reservoir fluid heterogeneity, and/or 4) contamination of the zones' end member oil samples. These conditions make it more challenging to accurately apply oil fingerprinting for production allocation.

Instead of producing a single best-fit model, the Markov Chain Monte Carlo (MCMC) approach provided herein may produce many (e.g., hundreds of thousands) 1-D models that simultaneously fit the data and satisfy available prior geological and engineering information. In this way, MCMC approach could provide an optimal solution to the allocation problems that satisfy the mathematical, geological, and engineering constraints. The MCMC approach may serve as an effective long term zonal allocation tool. Embodiments of estimating unknown proportions of a plurality of end-members in an unknown mixture are provided herein. One embodiment of a method of estimating unknown proportions of a plurality of end-members in an unknown mixture comprises receiving fingerprint data of a plurality of end-members and an unknown mixture comprising unknown proportions of the plurality of end-members; processing the fingerprint data of the plurality of end-members and the unknown mixture to generate peak height data of the plurality of end-members and the unknown mixture; and generating an estimate of the unknown proportions of the plurality of end-members in the unknown mixture by applying a Markov Chain Monte Carlo method (MCMC method) to the peak height data of the plurality of end-members and the unknown mixture.

For example, the MCMC method may be utilized for processing peak heights of oil fingerprinting data, such as gas chromatography data. The MCMC method provides probabilistic results of the contribution percentage of each reservoir/flowline to the commingled production flow, and it can: 1) accommodate non-normal distribution of the random errors to estimate the confidence interval, 2) manage non-linearity of the equations derived from the mixing, 3) distinguish subtle reservoir fluid differences against instrumentation noise, 4) minimize the effects of contamination, and/or 5) allow allocation with no limit of end numbers (discrete reservoir zones). Moreover, any pre-knowledge of the reservoir and production conditions (e.g., a zone known to dominate) could be integrated into the equations. Advantageously, the generated estimate from applying the MCMC method may be able to make it possible to use oil fingerprinting as a long term production allocation tool for downhole commingling. Of note, the term “contamination” in this disclosure refers to mixing or contact of oil samples with other components, such as, but not limited to, mixing or contact with chemicals used in drilling (e.g., downhole mixing of oil with drilling chemical(s)).

Advantageously, the MCMC may be utilized for unmixing of oil fingerprint data for production allocation. MCMC provides probabilistic estimation of the contribution percentage of each reservoir/flowline to the commingled production flow, accommodates the non-linear mixing behavior, and eliminates the need for lab mixed samples. Advantageously, the elimination of the need for lab mixed samples is especially helpful when there are limited end-member oils available (e.g., for downhole drill stem test (DST) & modular formation dynamics test (MDT) oil, and unconventional). Advantageously, any prior knowledge of the reservoir and production condition (e.g., the dominated contributing end-member information) may be incorporated in the calculation. In this way, MCMC may provide an even more accurate solution to the allocation problems that satisfy mathematical, geological, and/or engineering constraints. Advantageously, embodiments consistent with the present disclosure may be used to allocate the commingled production from multiple flowlines/pipelines and wells completed in multiple zones, and estimate the contribution from overlaying and underlaying formation for the hydraulically fractured lateral wells.

Advantageously, the MCMC method could accommodate non-normal distribution of the random errors for confidence interval estimation, and it could also minimize the effects of contamination. Furthermore, any pre-knowledge of the reservoir and production conditions (e.g., a zone known to dominate) could be integrated into the equations.

Advantageously, embodiments consistent with this disclosure may be utilized to generate short-term and long-term production forecasts. Advantageously, embodiments consistent with this disclosure may be utilized to generate more accurate production forecasts. The embodiments consistent with this disclosure may be utilized to forecast hydrocarbon production of a wellbore drilled in a conventional formation. The embodiments consistent with this disclosure may be utilized to forecast hydrocarbon production of a wellbore drilled in an unconventional formation. The production forecasts may enable better development planning, economic outlook, reserve estimates, and business decisions, reservoir management decisions (e.g., selection and execution of hydrocarbon recovery processes), especially for unconventional and tight rock reservoirs.

Advantageously, embodiments consistent with this disclosure may be utilized for wellbore intervention, for example, if the more accurate forecast indicates a decline in production. The early or preventative wellbore intervention may include a workover, fix or replace equipment (e.g., sandscreen, tubing, etc.), refracturing, change or adjust the hydrocarbon recovery process, etc.

Advantageously, embodiments consistent with this disclosure may be utilized to optimize productivity of a producing hydrocarbon bearing formation and drive reservoir management decisions. (1) As an example, embodiments consistent with this disclosure may be utilized to optimize well designs, including orientation of wellbores, casing points, completion designs, etc. (2) As another example, embodiments consistent with this disclosure may be utilized to identify landing zone (depth), geosteering to follow the landing zone, etc. For example, higher producers and their associated depths may be identified and utilized to drill a new wellbore to that identified associated depth. (3) As another example, the embodiments consistent with this disclosure may be utilized to control flow of fluids injected into or received from the formation, a wellbore, or any combination thereof. Chokes or well control devices that are positioned on the surface, downhole, or any combination thereof may be used to control the flow of fluid into and out. For example, surface facility properties, such as choke size, etc., may be identified for the high producers and that identified choke size may be utilized to control fluid into or out of a different wellbore.

Advantageous, embodiments consistent with this disclosure may be utilized in following: 1. Trouble shoot completion issues; 2. Proper production recording and planning; 3. Uncover production potential in existing assets and provide insight; 4. Evaluate the performance of workovers (e.g., acid job); 5. Calibrate the simulation model; and/or 6. Facilitate the taxing and accounting information for royalty payment, cost and profit share, or any combination thereof. Those of ordinary skill in the art may appreciate that there may be other advantages.

Of note, the principles of the present disclosure are not limited to production allocation. For example, the principles of the present disclosure may be utilized when dealing with flowback fluid in the context of hydraulic fracturing. For example, the principles of the present disclosure may be utilized with a produced fluid. For example, the principles of the present disclosure may be utilized with practically any fluid in which it would be advantageous to estimate unknown proportions of a plurality of end-members in the unknown mixture (i.e., the fluid).

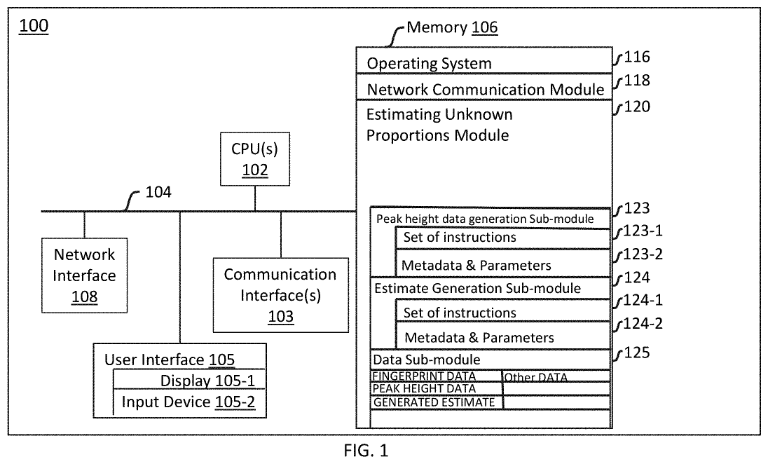

System—FIG. 1 is a block diagram illustrating a system of estimating unknown proportions of a plurality of end-members in an unknown mixture, such as a system 100 (such as a computing system or computer 100), in accordance with some embodiments. For example, the system 100 may be utilized for estimating unknown proportions of a plurality of end-members in an unknown mixture. While certain specific features are illustrated, those skilled in the art will appreciate from the present disclosure that various other features have not been illustrated for the sake of brevity and so as not to obscure more pertinent aspects of the embodiments disclosed herein.

To that end, the system 100 includes one or more processing units (CPUs) 102, one or more network interfaces 108 and/or other communication interfaces 103, memory 106, and one or more communication buses 104 for interconnecting these and various other components. The system 100 also includes a user interface 105 (e.g., a display 105-1 and an input device 105-2). The communication buses 104 may include circuitry (sometimes called a chipset) that interconnects and controls communications between system components. An operator can actively input information and review operations of system 100 using the user interface 105. User interface 105 can be anything by which a person can interact with system 100, which can include, but is not limited to, the input device 105-2 (e.g., a keyboard, mouse, etc.) or the display 105-1.

Memory 106 includes high-speed random access memory, such as DRAM, SRAM, DDR RAM or other random access solid state memory devices; and may include non-volatile memory, such as one or more magnetic disk storage devices, optical disk storage devices, flash memory devices, or other non-volatile solid state storage devices. Memory 106 may optionally include one or more storage devices remotely located from the CPUs 102. Memory 106, including the non-volatile and volatile memory devices within memory 106, comprises a non-transitory computer readable storage medium and may store data (e.g., (a) fingerprint data of a plurality of end-members and an unknown mixture comprising unknown proportions of the plurality of end-members, (b) peak height data of the plurality of end-members and the unknown mixture, (c) generated estimates, (d) geological data, perforation depth, perforation interval, reservoir temperature, reservoir pressure, or any combination thereof, etc.). In particular embodiments, the computer readable storage medium comprises at least some tangible devices, and in specific embodiments such computer readable storage medium includes exclusively non-transitory media.

In some embodiments, memory 106 or the non-transitory computer readable storage medium of memory 106 stores the following programs, modules and data structures, or a subset thereof including an operating system 116, a network communication module 118, and an estimating unknown proportions module 120.

The operating system 116 includes procedures for handling various basic system services and for performing hardware dependent tasks.

The network communication module 118 facilitates communication with other devices via the communication network interfaces 108 (wired or wireless) and one or more communication networks, such as the Internet, other wide area networks, local area networks, metropolitan area networks, and so on.

In some embodiments, the estimating unknown proportions module 120 executes the operations of the methods shown in the figures. The estimating unknown proportions module 120 may include data sub-module 125, which receives and handles data such as fingerprint data, etc. The fingerprint data may be received in a raw state. In one embodiment, the fingerprint instrument (e.g., a gas chromatography instrument) is coupled to a separate computing system via a wired connection and/or wireless connection, and the fingerprint data may be received at the system 100 from that separate computing system via a wired connection and/or wireless connection. Alternatively, or additionally, a user may input fingerprint data into the system 100 using the user interface 105 of the system 100. In this example, the user may retrieve the fingerprint data from the separate computing system that is coupled to the fingerprint instrument. Alternatively, the fingerprint data may be sent via a wired connection and/or wireless connection from one source to the system 100 (e.g., fingerprint data sent to the system 100 from a separate computing system at a vendor location).

A peak height data generation sub-module 123 contains a set of instructions 123-1 and accepts metadata and parameters 123-2 that will enable it to process the fingerprint data of the plurality of end-members and the unknown mixture to generate peak height data of the plurality of end-members and the unknown mixture. The sub-module 123 also aligns and indexes raw peaks in the fingerprint data of the plurality of end-members and the unknown mixture. In some embodiments, the peak height data may be output to an operator or to another system(s) via the user interface 105, the network communication module 118, a printer, the display 105-1, a data storage device, any combination of thereof, etc. In one embodiment, the fingerprint data may be processed using a commercially available product, such as, but not limited to, a product under the brand name ChromEdge from Weatherford (also known as Stratum Reservoir). In one embodiment, the fingerprint data may be processed using a commercially available product, such as, but not limited to, a product under the brand name Malcom from Schlumberger. Thus, the sub-module 123 may represent a commercially available tool or product in some embodiments.

An estimate generation sub-module 124 contains a set of instructions 124-1 and accepts metadata and parameters 124-2 that will enable it to generate an estimate of the unknown proportions of the plurality of end-members in the unknown mixture by applying a Markov Chain Monte Carlo method to the peak height data of the plurality of end-members and the unknown mixture. The sub-module 124 may also utilize geological data, perforation depth, perforation interval, reservoir temperature, reservoir pressure, or any combination thereof to constrain C when handling the misfit function.

Although specific operations have been identified for the sub-modules discussed herein, this is not meant to be limiting. Each sub-module may be configured to execute operations identified as being a part of other sub-modules, and may contain other instructions, metadata, and parameters that allow it to execute other operations of use in estimating unknown proportions. For example, any of the sub-modules may optionally be able to generate a display that would be sent to and shown on the user interface display 105-1. In addition, any of the data may be transmitted via the communication interface(s) 103 or the network interface 108 and may be stored in memory 106.

Method 200 is, optionally, governed by instructions that are stored in computer memory or a non-transitory computer readable storage medium (e.g., memory 106) and are executed by one or more processors (e.g., processors 102) of one or more computer systems. The computer readable storage medium may include a magnetic or optical disk storage device, solid state storage devices such as flash memory, or other non-volatile memory device or devices. The computer readable instructions stored on the computer readable storage medium may include one or more of: source code, assembly language code, object code, or another instruction format that is interpreted by one or more processors. In various embodiments, some operations in each method may be combined and/or the order of some operations may be changed from the order shown in the figures. For ease of explanation, method 200 is described as being performed by a computer system, although in some embodiments, various operations of method 200 are distributed across separate computer systems.

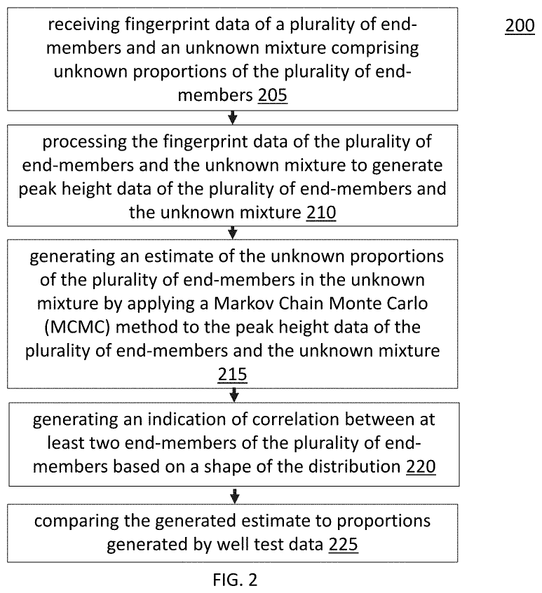

Turning to FIG. 2, this figure illustrates one embodiment of a method of estimating unknown proportions of a plurality of end-members in an unknown mixture, such as a method 200. The method 200 of FIG. 2 may be executed by the system 100 of FIG. 1, and a running example is utilized to discuss some portions of the method 200.

At 205, the method 200 includes receiving fingerprint data of a plurality of end-members and an unknown mixture comprising unknown proportions of the plurality of end-members. The term “receiving” includes practically any manner of receiving data, such as receiving, obtaining, accessing, etc. In one embodiment, fluid fingerprint data comprises chromatographic data (e.g., from a fingerprint instrument such as a gas chromatography instrument), isotope data (e.g., from a fingerprint instrument such as a mass spectrometer), water data (e.g., from a fingerprint instrument such as an ion chromatography instrument), or any combination thereof. In one embodiment, the plurality of end-members comprises at least four end-members. In one embodiment, the plurality of end-members comprises four end-members. In one embodiment, the plurality of end-members comprises five end-members. In one embodiment, the plurality of end-members comprises two to five end-members. In one embodiment, the unknown mixture comprises hydrocarbon, gas, water, or any combination thereof. The unknown mixture may be produced fluid from a wellbore, and the produced fluid may be produced by practically any hydrocarbon recovery process. In one embodiment, the fingerprint data may be received at the system 100 as explained hereinabove in connection with FIG. 1.

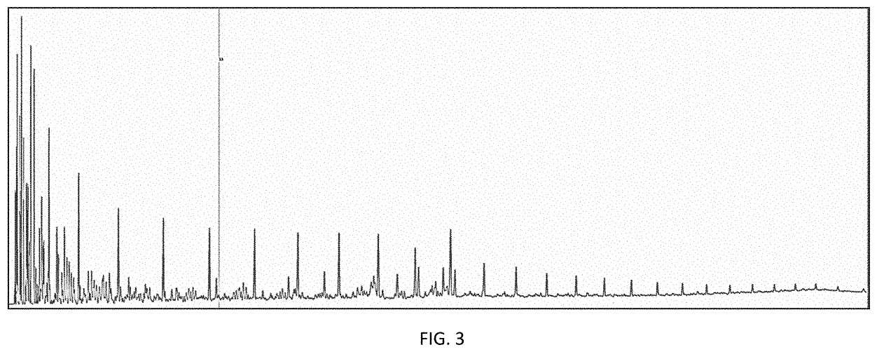

FIG. 3 illustrates one example of fingerprint data. In FIG. 3, the fingerprint data was generated by a gas chromatography instrument.

Moreover, turning to the running example, at 205, fingerprint dataA of end-memberA may be received. Similarly, at 205, fingerprint dataB of end-memberB may be received. Similarly, fingerprint dataUM of the unknown mixture may be received. The fingerprint data that is received may be chromatographic data, isotope data, water data, or any combination thereof. These examples are not meant to limit the principles of the present disclosure, and for example, the fingerprint data may be received in practically any way known to those of ordinary skill in the art.

At 210, the method 200 includes processing the fingerprint data of the plurality of end-members and the unknown mixture to generate peak height data of the plurality of end-members and the unknown mixture. In one embodiment, processing the fingerprint data of the plurality of end-members and the unknown mixture to generate the peak height data of the plurality of end-members and the unknown mixture comprises aligning and indexing raw peaks in the fingerprint data of each end-member and the unknown mixture. In one embodiment, alignment may be performed using a time shift alignment method, such as described in Zheng, Q X., et al. Automatic time-shift alignment method for chromatographic data analysis. Sci Rep 7, 256 (2017), which is incorporated by reference. In one embodiment, indexing may be performed using numerical indexing (e.g., name each peak one by one), Kovats indexing, or another type of indexing. The Kovats index is discussed in more detail in the following: Kovats, E. (1958). “Gas-chromatographische Charakterisierung organischer Verbindungen. Teil 1: Retentionsindices aliphatischer Halogenide, Alkohole, Aldehyde and Ketone”. Helv. Chim. Acta. 41 (7): 1915-32 and Rostad, C. E., et al., Kovats and lee retention indices determined by gas chromatography/mass spectrometry for organic compounds of environmental interest, Journal of High Resolution Chromatography, Volume 9, Issue 6, June 1986, pages 328-334, each of which is incorporated by reference. These are not exhaustive lists.

In one embodiment, the fingerprint data may be processed using a commercially available product, such as, but not limited to, a product under the brand name ChromEdge from Weatherford. In one embodiment, the fingerprint data may be processed using a commercially available product, such as, but not limited to, a product under the brand name Malcom from Schlumberger. This is not an exhaustive list.

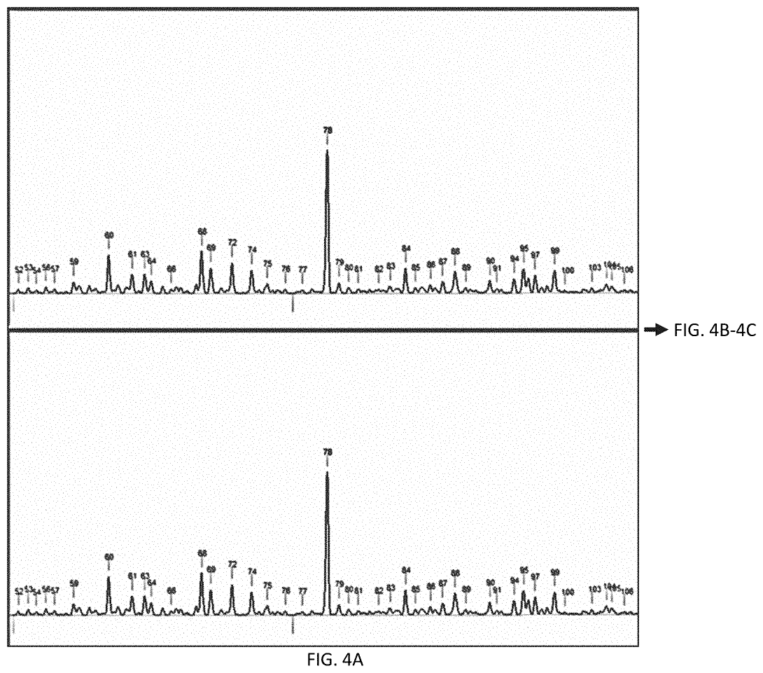

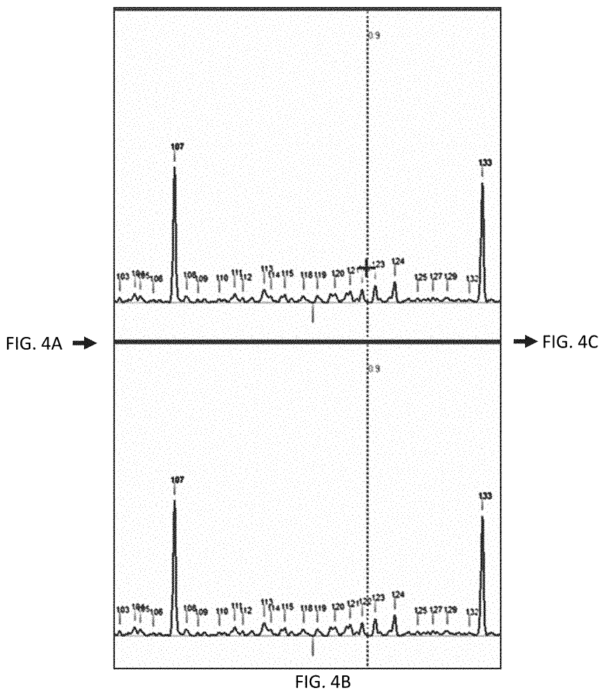

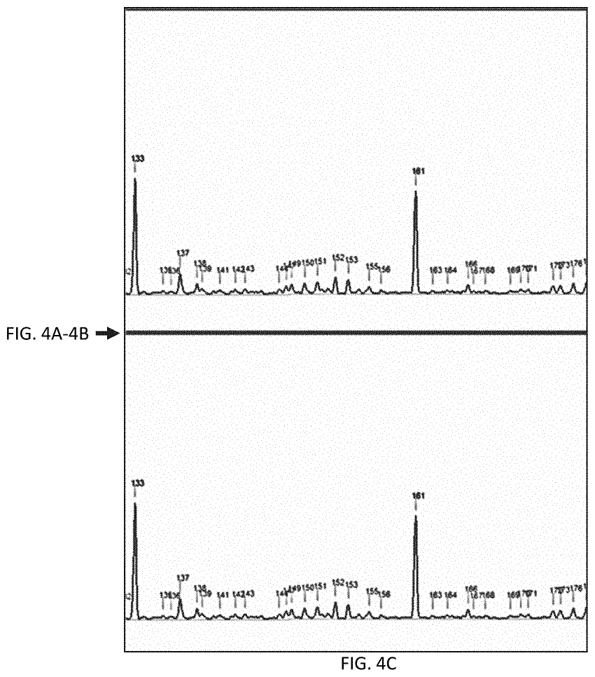

FIGS. 4A, 4B, and 4C illustrate examples of processing the fingerprint data of the plurality of end-members and the unknown mixture to generate peak height data of the plurality of end-members and the unknown mixture. Specifically, FIGS. 4A, 4B, and 4C (top) illustrate an example of peak alignment and FIGS. 4A, 4B, and 4C (bottom) illustrates an example of indexing.

Moreover, returning to the running example, at 210, the fingerprint dataA of end-memberA may be processed to generate peak height dataA, such as peak height dataA1, peak height dataA2, peak height dataA3, and peak height dataA4. Similarly, at 210, the fingerprint dataB of end-memberB may be processed to generate peak height dataB, such as peak height dataB 1, peak height dataB2, peak height dataB3, and peak height dataB4. Similarly, the fingerprint dataUM of the unknown mixture may be processed to generate peak height dataUM, such as peak height dataUM1, peak height dataUM2, peak height dataUM3, and peak height dataUM4. In this example, the peak height dataA for the end-memberA, the peak height dataB for the end-memberB, and the peak height dataUM for the unknown mixture include the same quantity (i.e., 4). These examples are not meant to limit the principles of the present disclosure, and for example, the fingerprint data may be processed in practically any way known to those of ordinary skill in the art that can generate peak height data.

At 215, the method 200 includes generating an estimate of the unknown proportions of the plurality of end-members in the unknown mixture by applying a Markov Chain Monte Carlo (MCMC) method to the peak height data of the plurality of end-members and the unknown mixture. In one embodiment, the Markov Chain Monte Carlo method described in the following item may be utilized: O Ruanaidh J. J. K., Fitzgerald W. J. (1996) Markov Chain Monte Carlo Methods. In: Numerical Bayesian Methods Applied to Signal Processing. Statistics and Computing. Springer, New York, N.Y., pp 69-95, which is incorporated by reference. In one embodiment, the the Markov Chain Monte Carlo method described in the following item may be utilized: Gilks, W. R.; Richardson, S.; Spiegelhalter, D. J. (1996). Markov Chain Monte Carlo in Practice. Chapman and Hall/CRC, which is incorporated by reference.

In one embodiment, applying the Markov Chain Monte Carlo method comprises using a misfit function, and the misfit function comprises:

$${misfit}_{i} = {\sum }_{j = 1}^{P} \vert \frac{{Y}_{ij} - {c}_{ik} {x}_{kj}}{{σ}_{i}} \vert$$

In the misfit function, σᵢ represents error of a fingerprint instrument, p represents total number of peaks, Y represents a matrix of peak heights of the unknown mixture, X represents a matrix of peak heights of a particular end-member, and C represents a matrix of unknown proportions of the unknown mixture. When the end member peak heights (X) are perfectly known, the problem of linear unmixing reduces to the inversion step. In this MCMC model, C_(i,k) are treated as free parameters. It forms an n-D model space, in which each point can be used to perform decomposition. The quality of the fitting for different mixtures is assessed by the misfit function above. The fingerprint instrument is the instrument that was utilized to generate the fingerprint data, such as the fingerprint data received at 205. Here, the L1-norm misfit function (sum of absolute deviations) is used to minimize the effect of outliers. Norm L1 Misfit is described further in the following: Claerbout, J. F., Muir, F., 1973. Robust modeling with erratic data. Geophysics 38 (5), 826-844, which is incorporated by reference. The MCMC method performs a “random walk” in the model space and saves a collection of model samples, which are chosen so that their corresponding modeled peak heights profiles give rise to reasonable misfit function.

The following option may also be utilized in applying the MCMC method. In one embodiment, Y=CX+Residue, and Residue represents an error of the fingerprint instrument, a random error, or any combination thereof. The physical unmixing problem can be expressed as the equation Y=CX+Residue. In one embodiment, C satisfies positivity and additivity constraints, and the positivity and additivity constraints comprise:

$$\{ \begin{matrix}{C}_{i , k} \geq 0 \\ {\sum }_{k = 1}^{n} {C}_{i , k} = 1\end{matrix}$$

In the constraints, i is a commingled sample index, j is a peak index, k is an end-member index, and n is a total number of endmembers. For example, due to physical considerations, the mix proportion vector C satisfies the positivity and additivity constraints. Besides these two constraints, we can incorporate other prior information as additional constraints from geological and engineering understanding. In one embodiment, C is constrained based on geological data (e.g., log data, permeability, porosity), perforation depth, perforation interval, reservoir temperature, reservoir pressure, or any combination thereof.

Turning to the generated estimate, in one embodiment, the generated estimate of the unknown proportions of the plurality of end-members in the unknown mixture is a distribution (e.g., a distribution of points). In one embodiment, the generated estimate of the plurality of end-members in the unknown mixture is a non-normal distribution of random errors (e.g., noise is not consistent with a normal distribution). In one embodiment, the generated estimate of the unknown proportions of the plurality of end-members in the unknown mixture is a single value (e.g., 0.25, 0.50, 0.23, 0.17, 0.88, etc.). In the context of single value, the single values generated for the plurality of end-members should sum up to about 1. In one embodiment, the generated estimate of the unknown proportions of the plurality of end-members in the unknown mixture comprises a distribution and a single value.

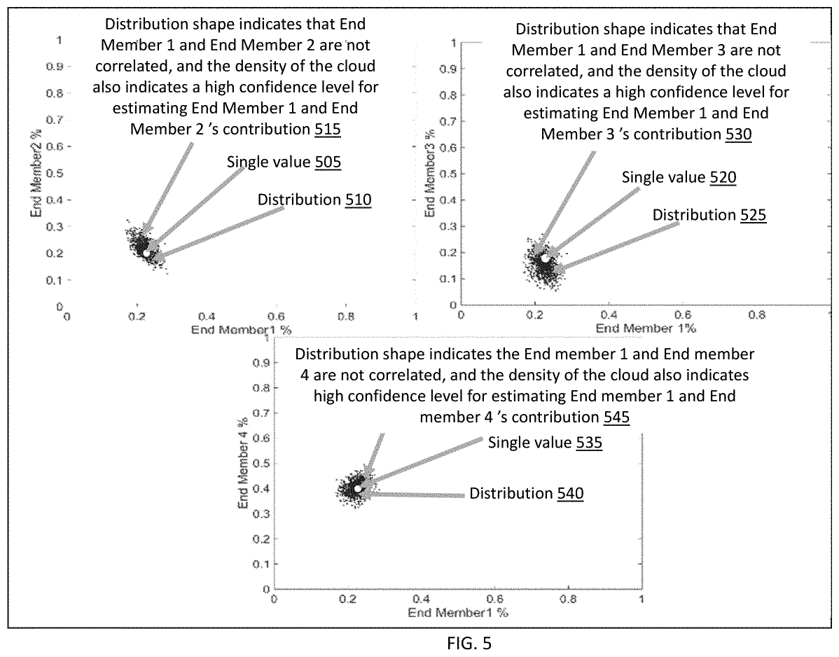

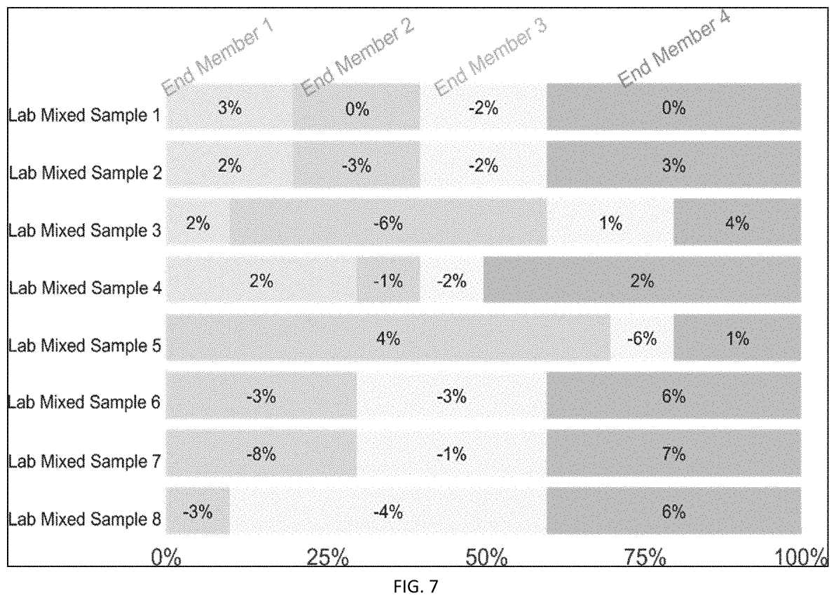

FIG. 5 illustrates examples of generating an estimate of the unknown proportions of the plurality of end-members in the unknown mixture by applying a Markov Chain Monte Carlo method to the peak height data of the plurality of end-members and the unknown mixture. Specifically, top left of FIG. 5 illustrates a generated estimate in the form of single value 505 (see big circle), as well as a generated estimate in the form of a distribution 510 (see points). The top right of FIG. 5 illustrates a generated estimate in the form of single value 520 (see big circle), as well as a generated estimate in the form of a distribution 525 (see points). The bottom of FIG. 5 illustrates a generated estimate in the form of single value 535 (see big circle), as well as a generated estimate in the form of a distribution 540 (see points). In FIG. 5, the single values illustrate the best fit and the points (dots) represent all the possible allocation results. The size of the cloud represents the uncertainty range for MCMC allocation results. MCMC results not only may provide the mix ratios, but also show how likely each mix ratio would be. It provides a much more comprehensive view of what may happen compared to conventional deterministic (linear regression) methods or “single-point estimate” analysis. Confidence intervals can be easily computed and allow the accuracy of different estimates to be quantified. FIG. 5 illustrates the MCMC results for 4 end member example in FIG. 7.

Moreover, returning to the running example, at 215, the MCMC method may be applied to the peak height dataA for end-memberA, the peak height dataB for end-memberB, and the peak height dataUM for the unknown mixture. MCMC method may lead to the following generated estimates in the form of single values: generated estimate of 0.25 for end-memberA in the unknown mixture, generated estimate of 0.75 for end-memberB in the unknown mixture, which total up to 1.00 (or 100%). For instance, the allocation of the end-memberA and the end-memberB in the unknown mixture is: the generated estimate of 0.25 for end-memberA in the unknown mixture and generated estimate of 0.75 for end-memberB in the unknown. In some embodiments, the generated estimates may be provided as visual output that may be viewable and/or printable by a user via the user interface 105 of the system 100 (e.g., visual output with single values, visual output in graph form such as in FIG. 5, etc.). These examples are not meant to limit the principles of the present disclosure, and for example, the estimate may be generated as a single value, a distribution, etc.

Optionally, at 220, the method 200 includes generating an indication of correlation between at least two end-members of the plurality of end-members based on a shape of the distribution. FIG. 5 illustrate three indications based on the shapes of the three distributions at 515, 530, and 545. In FIG. 5, the indications at 515, 530, and 545 indicate that the corresponding end-members are not correlated because the distribution of points is scattered. If correlated, the distribution of points would appear closer to a line shape. In some embodiments, the indication may be output as visual output that may be viewable and/or printable by a user via the user interface 105 of the system 100.

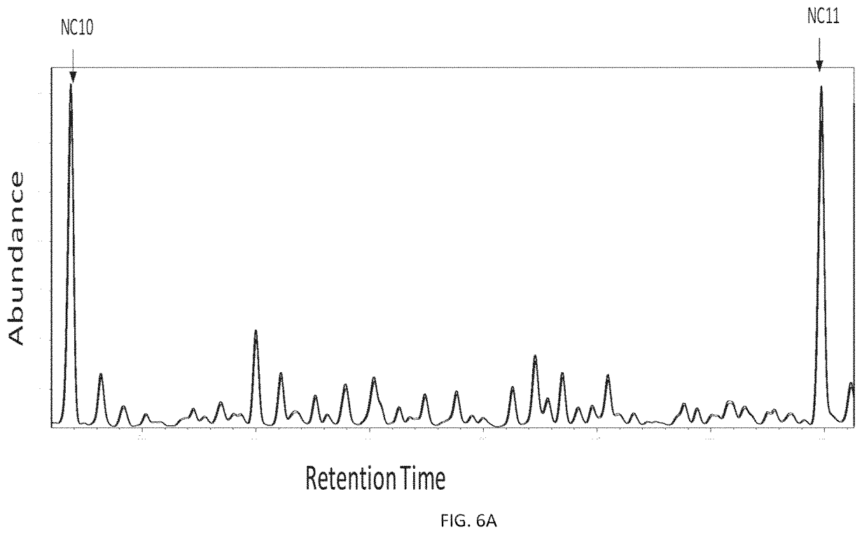

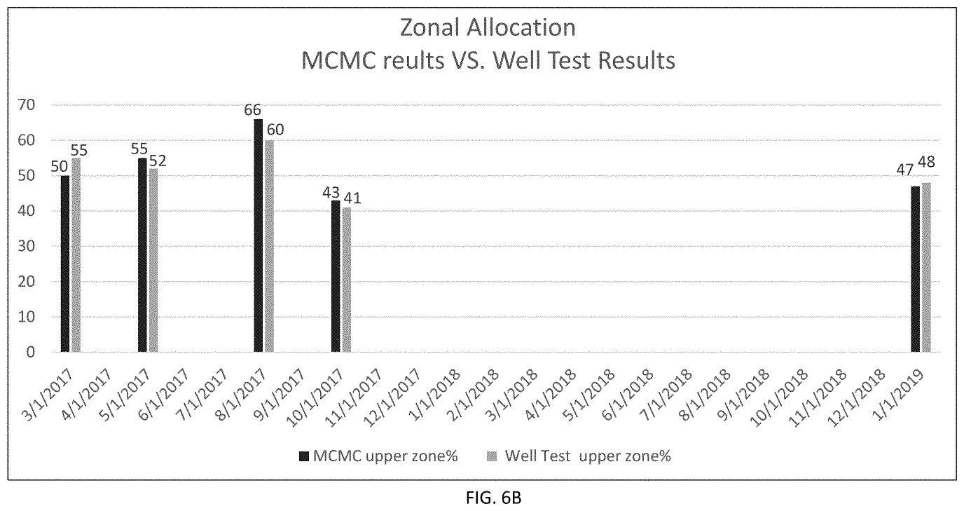

Optionally, at 225, the method 200 includes comparing the generated estimate to proportions generated by well test data. In one embodiment, the comparison indicates a difference of about 0% to about 6% or about 0% to about 10% or about 3% to about 6%. FIG. 6B illustrates examples of a difference of about 0% to about 6% based on a comparison of the generated estimate to proportions generated by well test data. In some embodiments, the difference may be output as visual output that may be viewable and/or printable by a user via the user interface 105 of the system 100.

### Example 1

One embodiment of the principles of the present disclosure has been validated on an intelligent well (IWC) with a dual-zone completion. The oils from two zones are extremely similar thus making it challenging to use least square regression to process the oil fingerprinting data. The similarity of the oils from 2 zones are illustrated in FIG. 6A. FIG. 6A illustrates Overlapped Gas Chromatograms of end-member oils from the dual-zone completed wells for NC10 to NC11 range, and the tight overlap suggested these two end-members are highly similar. This particular embodiment overcame the challenge and produced reliable results: MCMC allocation results are consistent with actual zonal well test measurements, with less than 6% difference from well test based allocations for all 5 tested samples collected over a period of time (illustrated in FIG. 6B). FIG. 6B illustrates validation of MCMC allocation results in an IWC well, in which geochemical samples were collected around the same time that zonal well tests were conducted, and the geochemical allocations are within 6% of the well test measurements. This demonstrates the reliability and accuracy of this MCMC approach.

### Example 2

Furthermore, as discussed hereinabove, FIG. 7 illustrates one example of validation using lab mixed samples with 4 end-members oil from the stacked reservoir of a single well. The size of the block represents the real lab mix ratios for each end-member, and the digital number in the block represents the errors between the MCMC calculated ratio and the real mixed ratio. The average error for 8 tested samples is less than 5%. The consistence between the true values with the calculated results proved the accuracy of this MCMC approach. In FIG. 5, the single values illustrate the best fit and the points (dots) represent all the possible allocation results. The size of the cloud represents the uncertainty range for MCMC allocation results. MCMC results not only may provide the mix ratios, but also show how likely each mix ratio would be. It provides a much more comprehensive view of what may happen compared to conventional deterministic (linear regression) methods or “single-point estimate” analysis. Confidence intervals can be easily computed and allow the accuracy of different estimates to be quantified. FIG. 5 illustrates the MCMC results for 4 end member example in FIG. 7.

In conclusion, those of ordinary skill in the art may appreciate the following: (1) Application of the Markov Chain Monte Carlo method for commingled production allocation and reservoir surveillance based on GC fingerprinting may be valuable and effective, especially in highly challenging offshore deepwater situations. (2) The MCMC method provides the probability distributions of the allocation results, and the non-normal distribution of error in each calculated ratio. It also allows incorporating geological and engineering constraints to the GC fingerprinting allocation process. The approach provides optimal solutions to allocation problems that satisfy the mathematical, geological and engineering constraints. (3) The accuracy and cost efficiency of oil fingerprinting production allocation allow reservoir engineers to monitor the production and zonal performance over long periods.

Numerous specific details are set forth in order to provide a thorough understanding of the subject matter presented herein. But it will be apparent to one of ordinary skill in the art that the subject matter may be practiced without these specific details. In other instances, well-known methods, procedures, components, and circuits have not been described in detail so as not to unnecessarily obscure aspects of the embodiments.

Although some of the various drawings illustrate a number of logical stages in a particular order, stages that are not order dependent may be reordered and other stages may be combined or broken out. While some reordering or other groupings are specifically mentioned, others will be obvious to those of ordinary skill in the art and so do not present an exhaustive list of alternatives. Moreover, it should be recognized that the stages could be implemented in hardware, firmware, software or any combination thereof.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

1. A method of estimating unknown proportions of a plurality of end-members in an unknown mixture, the method comprising:
receiving fingerprint data of a plurality of end-members and an unknown mixture comprising unknown proportions of the plurality of end-members;
processing the fingerprint data of the plurality of end-members and the unknown mixture to generate peak height data of the plurality of end-members and the unknown mixture; and
generating an estimate of the unknown proportions of the plurality of end-members in the unknown mixture by applying a Markov Chain Monte Carlo method to the peak height data of the plurality of end-members and the unknown mixture.

2. The method of claim 1, wherein the generated estimate of the unknown proportions of the plurality of end-members in the unknown mixture is a distribution, a single value, or a distribution and a single value.

3. The method of claim 2, wherein the generated estimate of the plurality of end-members in the unknown mixture is a non-normal distribution of random errors.

4. The method of claim 2, further comprising generating an indication of correlation between at least two end-members of the plurality of end-members based on a shape of the distribution.

5. The method of claim 1, wherein processing the fingerprint data of the plurality of end-members and the unknown mixture to generate the peak height data of the plurality of end-members and the unknown mixture comprises aligning and indexing raw peaks in the fingerprint data of the plurality of end-members and the unknown mixture.

6. The method of claim 1, wherein applying the Markov Chain Monte Carlo method comprises using a misfit function, and wherein the misfit function comprises:
$${misfit}_{i} = {\sum }_{j = 1}^{P} \vert \frac{{Y}_{ij} - {c}_{ik} {x}_{kj}}{{σ}_{i}} \vert$$
wherein σᵢ represents error of a fingerprint instrument, p represents total number of peaks, Y represents a matrix of peak heights of the unknown mixture, X represents a matrix of peak heights of a particular end-member, and C represents a matrix of unknown proportions of the unknown mixture.

7. The method of claim 6, wherein Y=CX+Residue, and wherein Residue represents an error of the fingerprint instrument, a random error, or any combination thereof.

8. The method of claim 6, wherein C satisfies positivity and additivity constraints, and wherein the positivity and additivity constraints comprise:
$$\{ \begin{matrix}{C}_{i , k} \geq 0 \\ {\sum }_{k = 1}^{n} {C}_{i , k} = 1\end{matrix}$$
wherein i is a commingled sample index, j is a peak index, k is an end-member index, and n is a total number of end-members.

9. The method of claim 6, wherein C is constrained based on geological data, perforation depth, perforation interval, reservoir temperature, reservoir pressure, or any combination thereof.

10. The method of claim 1, further comprising comparing the generated estimate to proportions generated by well test data.

11. A system comprising:
a processor; and
a memory communicatively connected to the processor, the memory storing computer-executable instructions which, when executed, cause the processor to perform a method of estimating unknown proportions of a plurality of end-members in an unknown mixture, the method comprising:
receiving fingerprint data of a plurality of end-members and an unknown mixture comprising unknown proportions of the plurality of end-members;
processing the fingerprint data of the plurality of end-members and the unknown mixture to generate peak height data of the plurality of end-members and the unknown mixture; and
generating an estimate of the unknown proportions of the plurality of end-members in the unknown mixture by applying a Markov Chain Monte Carlo method to the peak height data of the plurality of end-members and the unknown mixture.

12. The system of claim 11, wherein the generated estimate of the unknown proportions of the plurality of end-members in the unknown mixture is a distribution, a single value, or a distribution and a single value.

13. The system of claim 12, wherein the executable instructions which, when executed, cause the processor to generate an indication of correlation between at least two end-members of the plurality of end-members based on a shape of the distribution.

14. The system of claim 11, wherein processing the fingerprint data of the plurality of end-members and the unknown mixture to generate the peak height data of the plurality of end-members and the unknown mixture comprises aligning and indexing raw peaks in the fingerprint data of the plurality of end-members and the unknown mixture.

15. The system of claim 11, wherein applying the Markov Chain Monte Carlo method comprises using a misfit function, and wherein the misfit function comprises:
$${misfit}_{i} = {\sum }_{j = 1}^{P} \vert \frac{{Y}_{ij} - {c}_{ik} {x}_{kj}}{{σ}_{i}} \vert$$
wherein σᵢ represents error of a fingerprint instrument, p represents total number of peaks, Y represents a matrix of peak heights of the unknown mixture, X represents a matrix of peak heights of a particular end-member, and C represents a matrix of unknown proportions of the unknown mixture.

16. The system of claim 15, wherein Y=CX+Residue, and wherein Residue represents an error of the fingerprint instrument, a random error, or any combination thereof.

17. The system of claim 15, wherein C satisfies positivity and additivity constraints, and wherein the positivity and additivity constraints comprise:
$$\{ \begin{matrix}{C}_{i , k} \geq 0 \\ {\sum }_{k = 1}^{n} {C}_{i , k} = 1\end{matrix}$$
wherein i is a commingled sample index, j is a peak index, k is an end-member index, and n is a total number of end-members.

18. The system of claim 15, wherein C is constrained based on geological data, perforation depth, perforation interval, reservoir temperature, reservoir pressure, or any combination thereof.

19. The system of claim 11, wherein the executable instructions which, when executed, cause the processor to compare the generated estimate to proportions generated by well test data.

20. A computer readable storage medium having computer-executable instructions stored thereon which, when executed by a computer, cause the computer to perform a method of estimating unknown proportions of a plurality of end-members in an unknown mixture, the method comprising:
receiving fingerprint data of a plurality of end-members and an unknown mixture comprising unknown proportions of the plurality of end-members;
processing the fingerprint data of the plurality of end-members and the unknown mixture to generate peak height data of the plurality of end-members and the unknown mixture; and
generating an estimate of the unknown proportions of the plurality of end-members in the unknown mixture by applying a Markov Chain Monte Carlo method to the peak height data of the plurality of end-members and the unknown mixture.
